(19) United States
(12) Patent Application Publication
(10) Pub. No.: US 2017/0021049 A1
(43) Pub. Date: Jan. 26, 2017

(54) AIR PURIFIER FOR BRINGING GAS INTO CONTACT WITH PLASMA-TREATED LIQUID

(71) Applicant: Panasonic Intellectual Property Management Co., Ltd., Osaka (JP)

(72) Inventors: TATSUSHI OHYAMA, Osaka (JP); HIROKO MURAYAMA, Osaka (JP); MASAHIRO ISEKI, Osaka (JP)

(21) Appl. No.: 15/206,473

(22) Filed: Jul. 11, 2016

(30) Foreign Application Priority Data
Jul. 24, 2015 (JP) 2015-147168

(51) Int. Cl. A61L 9/22; B01D 53/32; A61L 9/16

(52) U.S. Cl. CPC A61L 9/22; A61L 9/16; B01D 53/32; A61L 2209/14; B01D 2259/818

(57) ABSTRACT

An air purifier includes a case that includes an air inlet and an air outlet, a tank that is arranged in the case and stores liquid, a fan that causes an air flow from the air inlet to the air outlet to be produced, a partition that is arranged in the case and includes a filter that causes the air flow and the liquid to come into contact with each other while allowing the air flow to pass through the filter, and a plasma generator that includes a pair of electrodes arranged in a first space between the air inlet and the partition and a power supply and generates plasma so that the plasma comes into contact with the liquid.

### BACKGROUND

1. Technical Field

The present disclosure relates to an air purifier.

2. Description of the Related Art

In known conventional techniques, water in a water tank of a humidifying unit of an air processing apparatus is purified using electric discharge.

For example, Japanese Patent No. 4656138 discloses an air processing apparatus including an electric discharge processing unit, which performs electric discharge to produce an active species, and an air purifying means, which includes a filter and a deodorizing member. In the air processing apparatus, air that contains the active species is supplied to the air purifying means and water in a water tank.

### SUMMARY

An air purifier according to an aspect of the present disclosure includes: a case having an air inlet and an air outlet; a tank that stores liquid, the tank disposed in the case; a fan that produces an air flow from the air inlet to the air outlet, a partition disposed in the case, the partition including a filter through which the air flow comes into contact with the liquid, and a plasma generator that generates plasma which is to be in contact with the liquid, the plasma generator including a pair of electrodes which is disposed in a first space between the air inlet and the partition and a power supply which applies a voltage between the pair of electrodes.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A illustrates an example of a structure of an air purifier according to a first embodiment;

FIG. 1B illustrates another example of a structure of an air purifier according to the first embodiment;

FIG. 2A presents a front view and a side view that illustrate an example of a structure of a partition in the air purifier according to the first embodiment;

FIG. 2B presents a front view and a side view that illustrate another example of a structure of a partition in the air purifier according to the first embodiment;

FIG. 3 is a flow chart that illustrates an example of operation of the air purifier according to the first embodiment;

FIG. 4 illustrates an example of temporal change in hexanal concentration in a case where the air purifier according to the first embodiment is used;

FIG. 5 illustrates an example of temporal change in carbon dioxide concentration in the case where the air purifier according to the first embodiment is used;

FIG. 6 illustrates a structure of an air purifier according to a reference example of the first embodiment;

FIG. 7 illustrates an example of a structure of an air purifier according to a second embodiment;

FIG. 8 presents a front view and a side view that illustrate an example of a structure of a partition in the air purifier according to the second embodiment;

FIG. 9 illustrates an example of a structure of an air purifier according to a third embodiment;

FIG. 10 illustrates an example of a structure of an air purifier according to a fourth embodiment;

FIG. 11 illustrates an example of a structure of an air purifier according to a fifth embodiment;

FIG. 12 illustrates an example of a structure of another air purifier according to the fifth embodiment; and

FIG. 13 illustrates results of sensing concentrations of odorous substances in the proximity of an air outlet according to each of an example and a reference example using a volatile organic compound (VOC) sensor.

### DETAILED DESCRIPTION

### Overview of Embodiments

An air purifier according to an aspect of the present disclosure includes a case that includes an air inlet and an air outlet, a fan that produces an air flow so that gas is taken into the case through the air inlet or so that gas is discharged outside the case through the air outlet, a tank that is arranged in the case and stores liquid, a plasma generator that includes a pair of electrodes and a power supply to apply a voltage between the pair of electrodes and generates plasma so that the plasma comes into contact with the liquid stored in the tank, and a filter that is arranged so as to partition an inside of the case into a first space including the air inlet and a second space including the air outlet and causes the liquid stored in the tank and the gas in the case to come into contact with each other. The air purifier may further include a guide arranged along an edge of the filter. The plasma generator may be arranged in the first space.

Thus, the gas containing a substance to be decomposed can dissolve in the liquid through the filter to come into contact with an active species in the liquid, thereby being decomposed and/or deodorized. Accordingly, the air purifier can purify the air with efficiency.

The guide can inhibit the gas from moving from the first space to the second space without passing through the partition. As a result, much of the gas taken in from the air inlet can pass through the filter and thus, a substance contained in the gas can be decomposed with higher efficiency.

When reactive gas, such as ozone gas or NOₓ, is produced using plasma, the reactive gas dissolves in the liquid through the filter. Thus, the reactive gas can be inhibited from flowing outside the apparatus.

For example, 90% or more of the gas that moves from the first space to the second space may pass through the filter.

Thus, most part of the air taken into the case through the air inlet and most part of the reactive gas produced by the plasma generator can pass through the filter. Accordingly, contact between the reactive gas and the substance to be decomposed, and/or contact between the liquid containing the active species and the substance can be promoted. As a result, air can be purified with high efficiency.

For example, the size of a gap between the guide and the filter, the size of a gap between the guide and an inside face of the case, and the size of a gap between the guide and the liquid stored in the tank may be equal to or smaller than an average value of pore diameters of the filter.

Thus, most part of the air taken into the case through the air inlet and most part of the reactive gas produced by the plasma generator can pass through the filter. Accordingly, contact between the reactive gas and the substance to be decomposed, and/or contact between the liquid that contains the active species and the substance can be promoted. As a result, air can be purified with high efficiency.

For example, the guide may be a frame provided along an edge of the filter. The outside edge of the frame may be in contact with the inside face of the case, and/or may be in contact with the liquid stored in the tank.

When the gap between the outside edge of the guide and the inside face of the case is absent or small, the gas can be inhibited from moving from the first space to the second space through the gap. When the gap between the outside edge of the guide and the surface of the liquid is absent or small, the gas can be inhibited from moving from the first space to the second space through the gap. That is, most part of the air taken into the case through the air inlet and most part of the gas produced by the plasma generator can pass through the filter. Thus, air can be purified with efficiency.

For example, part of the edge of the filter may be arranged so as to be in contact with the liquid in the tank. The guide may be in contact with the inside face of the case along the part other than the above-mentioned part of the edge of the filter.

Thus, most part of the gas taken into the case through the air inlet and most part of the gas produced by the plasma generator can pass through the filter. Accordingly, air can be purified with efficiency.

For example, the filter may be arranged so as to be spaced from the liquid in the tank. The guide may be a loop-like frame provided for the entire perimeter of the edges of the filter. Part of the frame may be positioned so as to block a gap between the filter and the liquid in the tank.

Thus, most part of the air taken into the case through the air inlet and most part of the gas produced by the plasma generator can pass through the filter. Accordingly, air can be purified with efficiency.

For example, the air purifier may further include a gas supplying pump that supplies gas inside the first space or outside the case to the proximity of the pair of electrodes.

Thus, the plasma generator can generate plasma in a bubble produced using the supplied gas. Accordingly, power for vaporizing the liquid through electric discharge can be reduced and the power of the plasma generator can be utilized for the generation of plasma effectively. As a result, the power consumption can be reduced.

Embodiments are described in detail below with reference to the drawings.

All of the embodiments described below present comprehensive or specific examples. The values, shapes, materials, constituents, arrangements of the constituents, connection forms of the constituents, steps, sequence of the steps, and the like that are indicated below in the embodiments are examples and are not intended to limit the present disclosure. Among the constituents of the embodiments below, the constituents not recited in the independent aspect of the present disclosure, which indicates the most superordinate concept, can be explained as given constituents.

### First Embodiment

### [1. Structure of Air Purifier]

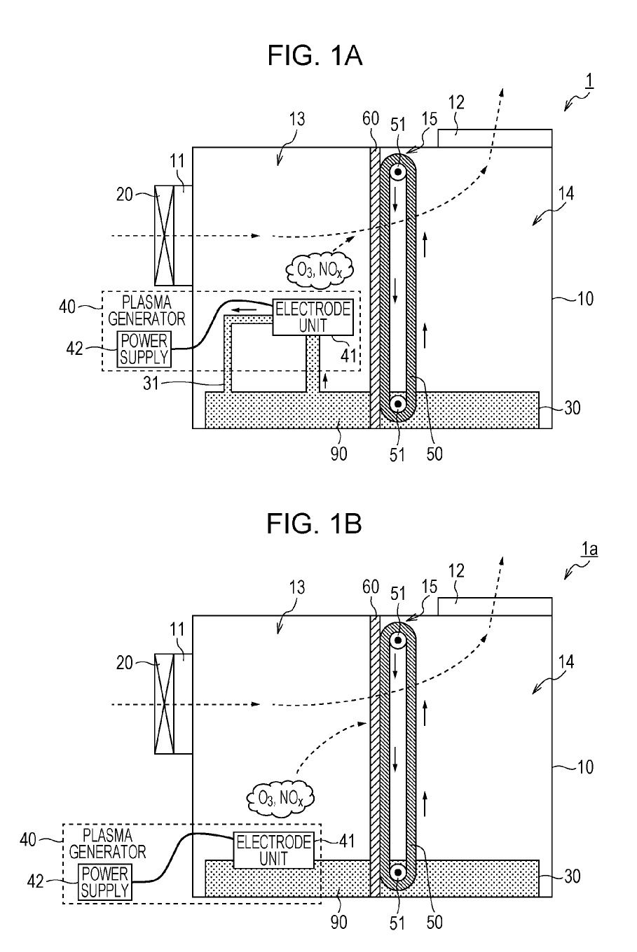

Structures of air purifiers 1 and 1a according to a first embodiment are described with reference to FIGS. 1A and 1B. FIG. 1A illustrates a structure of the air purifier 1 according to the present embodiment. FIG. 1B illustrates a structure of the air purifier 1a according to the present embodiment.

The air purifiers 1 and 1a each decompose a substance contained in air using plasma. For example, the air purifiers 1 and 1a each generate plasma to produce an active species in liquid 90, and take the air containing the substance into liquid 90. Thus, the active species and the substance can react together in the liquid 90, thereby enabling the substance to be decomposed.

Examples of the substance to be decomposed include a hazardous substance, a pollutant, and an odorous substance. The hazardous substance is for example, a chemical substance hazardous to humans or ecosystems. The odorous substance is for example, a chemical substance that causes an offensive odor. The substance to be decomposed may be for example, a fine particle or a microbe. Examples of the fine particle include pollen and house dust.

The air purifiers 1 and 1a are each utilized as an air cleaner for example. In this case, for example, the air purifiers 1 and 1a are each arranged in a predetermined space, such as a room, and purify the air in the space.

For another example, the air purifiers 1 and 1a are each utilized as a deodorizing apparatus or a sterilizer. In this case, the air purifiers 1 and 1a may each be provided in for example, an apparatus that preserves or cooks food, such as a refrigerator, a microwave oven, or a fryer. For another example, the air purifiers 1 and 1a may each be provided in an air conditioner, a humidifier, a laundry machine, a dish washer, or a vehicle.

### [2. Various Components of Air Purifier]

Various components of the air purifiers 1 and 1a are described in detail. Each component of the air purifier 1 illustrated in FIG. 1A is described first.

As illustrated in FIG. 1A, the air purifier 1 includes a case 10, a fan 20, a tank 30, a plasma generator 40, a filter 50, and a guide 60. The filter 50 is an example of a gas-liquid contact member.

### [2-1. Case]

The case 10 forms the outline of the air purifier 1. For example, the case 10 is formed of a resin material, such as plastic, and/or a metallic material. The case 10 may have any shape. As illustrated in FIG. 1A, the case 10 has an air inlet 11 and an air outlet 12.

The air inlet 11 is an opening for taking in air from the outside of the case 10 to the inside thereof. The air outlet 12 is an opening for discharging the air in the case 10 to the outside thereof. The air is taken into the case 10 from the air inlet 11, and after passing through the filter 50, is discharged from the air outlet 12 to the outside of the case 10 (see the dashed-line arrows in FIG. 1A).

In the example illustrated in FIG. 1A, the air inlet 11 is provided on a side face of the case 10 and the air outlet 12 is provided on an upper face of the case 10. With this configuration, since a distance between the air inlet 11 and the air outlet 12 is long, space for placing the filter 50 can be ensured between the air inlet 11 and the air outlet 12, and air can be caused to flow smoothly from the air inlet 11 to the air outlet 12.

The positions in which the air inlet 11 and the air outlet 12 are provided are not limited to the above-described examples. For example, the air inlet 11 and the air outlet 12 may each be provided on any of the upper face, side faces, and lower face of the case 10. For example, the air inlet 11 may be provided on the upper face of the case 10 and the air outlet 12 may be provided on the side face of the case 10. For another example, both of the air inlet 11 and the air outlet 12 may be provided on one face selected from the side faces, upper face, and lower face. Although in the example illustrated in FIG. 1A, the case 10 has the single air inlet 11 and the single air outlet 12, the case 10 may have a plurality of air inlets 11 and/or may have a plurality of air outlets 12. The air outlet 12 may be provided with a filter, which absorbs moisture in the air.

The inner space of the case 10 is partitioned by the filter 50 into a first space 13 and a second space 14. The first space 13 is connected to the air inlet 11 and the second space 14 is connected to the air outlet 12.

In the example illustrated in FIG. 1A, the guide 60 is arranged so as to block a gap 15 between the filter 50 and the inside face of the case 10. For example, the guide 60 is a frame formed along an edge of the filter 50.

In the example illustrated in FIG. 1A, the inner space of the case 10 is partitioned almost completely by the filter 50 and the guide 60 into the first space 13 and the second space 14. In other words, in the example illustrated in FIG. 1A, the partition that partitions the inner space of the case 10 into the first space 13 and the second space 14 is constituted of the filter 50 and the guide 60.

### [2-2. Fan]

The fan 20 is an example of an air blower that produces an air flow from the air inlet 11 to the air outlet 12. That is, the fan 20 produces an air flow from the first space 13 to the second space 14.

The fan 20 is provided on at least one of the air inlet 11 and the air outlet 12. When the fan 20 is provided on the air inlet 11, the fan 20 takes in gas into the case 10 through the air inlet 11. Thus, for example, a pressure difference is caused between the inside and outside of the case 10 and the gas is discharged outside the case 10 through the air outlet 12. When the fan 20 is provided on the air outlet 12, the fan 20 discharges the gas outside the case 10 through the air outlet 12. Thus, for example, a pressure difference is caused between the inside and outside of the case 10 and gas is taken into the case 10 through the air inlet 11. Each of these is an example of the “fan that produces an air flow from the air inlet to the air outlet” according to the present disclosure.

Although in the example illustrated in FIG. 1A, the fan 20 is provided on the air inlet 11, the fan 20 may be provided on the air outlet 12 only or may be provided on each of the air inlet 11 and the air outlet 12. For example, when the fan 20 is provided on each of the air inlet 11 and the air outlet 12, the fans 20 can cause air to flow more smoothly from the air inlet 11 to the air outlet 12. The fan 20 may be provided inside the case 10.

### [2-3. Tank]

The tank 30 is arranged in the case 10. The tank 30 stores the liquid 90. For example, the tank 30 is a box-like body, which has an open upper face like a tray, and is arranged on the bottom face of the case 10.

At first, the liquid 90 is pure water, for example. After generation of plasma, the liquid 90 contains the active species. Examples of the active species include a hydroxyl radical (OH), a hydrogen radical (H), an oxygen radical (O), a superoxide anion (O₂—), a monovalent oxygen ion (O—), and hydrogen peroxide (H₂O₂). In addition, when a reactive gas containing a nitrogen monoxide (NO) gas and/or a nitrogen dioxide (NO₂) gas comes into contact with the liquid 90 to dissolve thereinto, the liquid 90 may contain nitrous acid (HNO₂) as an active species, for example. These active species can decompose a substance by oxidation or reduction. For example, when the substance is an odorous substance, the active species can deodorize the odorous substance.

The liquid 90 may be water containing a compound, instead of pure water. For example, the liquid 90 may contain a compound for promoting the decomposition of the substance.

As illustrated in FIG. 1A, piping 31 connected to the tank 30 may be provided. The piping 31 is provided with part of the plasma generator 40. For example, the plasma generator 40 produces an active species in the liquid 90 in the piping 31, and the active species spreads in the tank 30 through the piping 31.

The piping 31 is constituted of for example, a tubular member, such as a pipe, a tube, or a hose. In the example illustrated in FIG. 1A, the piping 31 is provided on the upper side of the tank 30. For example, a pump (not illustrated) may be provided on a path of the piping 31 to circulate the liquid 90 in a direction (see the solid-line arrows in FIG. 1A).

The tank 30 and the piping 31 are each formed of for example, a resin material or a metallic material. When the tank 30 and the piping 31 are each formed of a metallic material, a process of plating and/or coating may be performed on surfaces thereof so as to prevent rust.

As illustrated in FIG. 1B, the air purifier 1a may omit the piping 31. In this case, the plasma generator 40 generates plasma so that the plasma comes into contact with the liquid 90 in the tank 30. For example, both of a pair of electrodes of the plasma generator 40 may be arranged in the liquid 90. For another example, one or both of the pair of electrodes may be arranged in the atmosphere so as not to come into contact with the liquid 90. In this case, for example, the plasma generator 40 generates plasma in the atmosphere to produce an active species in a gas such that the gas containing the active species comes into contact with the liquid 90. Thus, the air purifier 1a can be made with a simple structure.

### [2-4. Plasma Generator]

The plasma generator 40 generates plasma so as to produce an active species in the liquid 90. For example, the plasma generator 40 generates plasma so that the plasma comes into contact with the liquid 90 stored in the tank 30. For example, the plasma generator 40 is provided on a path of the piping 31 and generates plasma in the liquid 90 in the piping 31.

For example, the plasma generator 40 includes an electrode unit 41, which includes the pair of electrodes, and a power supply 42, which applies a voltage between the pair of electrodes. The electrodes forming the pair are spaced from each other and exposed in the piping 31. For example, the power supply 42 applies a negative-polarity high-voltage pulse of 2 to 50 kV/cm and 100 Hz to 100 kHz between the pair of electrodes to causes electric discharge in the liquid 90.

Because of evaporation of the liquid 90 caused by the energy of the electric discharge, and vaporization of the liquid 90 caused by shock waves with the electric discharge, a bubble is produced near at least one of the pair of electrodes in the liquid 90 in the piping 31. The plasma generator 40 generates plasma in the bubble so as to produce an active species in the liquid 90. Accordingly, the active species exist in abundance near the electrode unit 41, thus efficiently decomposing a substance therenear.

In the example illustrated in FIG. 1A, the plasma generator 40 is at least partly provided in a space between the air inlet 11 and the filter 50, that is, in the first space 13. When the plasma generator 40 generates plasma, gas with high reactivity, such as ozone gas (O₃) or NO_{X} (e.g., NO, NO₂), that is, reactive gas is produced. For example, the reactive gas produced in the first space 13 dissolves in the liquid 90 in the filter 50.

### [2-5. Filter]

The filter 50 is provided between the air inlet 11 and the air outlet 12. The liquid 90 supplied from the tank 30 and the air in the case 10 come into contact with each other through the filter 50. The filter 50 is arranged so as to cross the air flow formed by the fan 20. The filter 50 is arranged so as to partition the space inside the case 10 into the first space 13 and the second space 14.

For example, the filter 50 is provided so that the gap 15 between the filter 50 and the side face of the case 10 and the gap 15 between the filter 50 and the upper face of the case 10 are small. Thus, most part of the air taken in from the air inlet 11 can pass through the filter 50. As described below, the guide 60 may be provided so as to block the gap 15.

The filter 50 may be a member that increases the area of the liquid 90 in contact with the air. For example, the filter 50 may be a porous member formed of stainless steel or a chemical. The porous member is a porous plate for example, which includes a plurality of minute pores. The average value of the pore diameters of the filter 50 is for example, equal to or smaller than several millimeters. The average value of the pore diameters of the filter 50 is obtained by for example, averaging the diameters of a plurality of pores that appear on a given cross section of the filter 50. When the shape of the pore is not circular, the diameter of the pore corresponds to the diameter of a circle having the same area as the area of the pore. When the pores catch air, the air and the liquid 90 can easily come into contact with each other. The filter 50 can catch the gas produced by the plasma generator 40 in addition to the air taken into the case 10.

For another example, the filter 50 may be a fabric-like member with breathability and water absorbability. The filter 50 may have a plurality of napped portions so as to increase the surface area.

In the example illustrated in FIG. 1A, the filter 50 has a wide loop belt shape and is stretched between a pair of pulleys 51. The filter 50 is rotated by the pair of pulleys 51.

In the example illustrated in FIG. 1A, while part of the filter 50 is soaked in the liquid 90 in the tank 30, the filter 50 is rotated by the pulleys 51 (see the solid-line arrows in FIG. 1A). Thus, the filter 50 is exposed to the air in a state where the overall filter 50 contains the liquid 90. Thus, a substance contained in the air can come into contact with the active species in the liquid 90 through the filter 50, and thus can be decomposed by reaction with the active species.

The substance that has been taken into the liquid 90 in the filter 50 but has not been decomposed may be conveyed into the tank 30 with the rotation of the pulleys 51 to be decomposed in the tank 30 or the piping 31. The liquid 90 in the tank 30 and the piping 31 may contain a larger amount of the active species than the liquid 90 in the filter 50.

### [2-6. Guide]

The guide 60 is arranged along an edge of the filter 50 so as to block the gap 15. The guide 60 inhibits gas from flowing from the first space 13 to the second space 14 through the gap 15. For example, the guide 60 causes 90% or more of the gas that moves from the first space 13 to the second space 14 to pass through the filter 50.

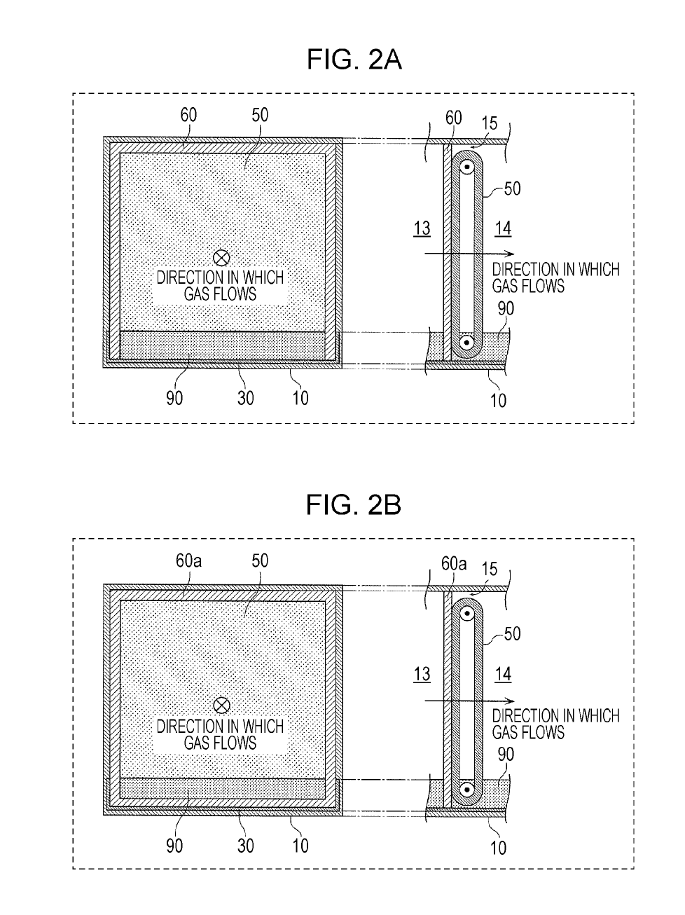

FIG. 2A illustrates an example of the partition of the air purifier 1. The left-hand illustration of FIG. 2A is a front view when the partition is viewed in a direction in which gas flows. The right-hand illustration of FIG. 2A is a side view when the partition is viewed from a side. The partition in FIG. 2A includes the filter 50 and the guide 60.

The guide 60 is arranged so as to come into contact with a face of the filter 50, which is on the side of the air inlet 11, so that the gap 15 between the filter 50 and the case 10 is blocked.

The size of the gap between the guide 60 and the filter 50 is equal to or smaller than the average value of the pore diameters of the filter 50. When for example, a plurality of gaps are present between the guide 60 and the filter 50, all of the plurality of gaps are equal to or smaller than the average value of the pore diameters of the filter 50.

The size of the gap between the guide 60 and the inside face of the case 10, and/or the size of the gap between the guide 60 and the liquid 90 stored in the tank 30 is/are equal to or smaller than the average value of the pore diameters of the filter 50. When for example, a plurality of gaps are present between the guide 60 and the inside face of the case 10, and/or when a plurality of gaps are present between the guide 60 and the liquid 90, the size of each of the plurality of gaps is equal to or smaller than the average value of the pore diameters of the filter 50.

In the example illustrated in FIG. 2A, the gas that flows from the first space 13 to the second space 14 can pass through the pores of the filter 50, the gap between the filter 50 and the guide 60, the gap between the guide 60 and the inside face of the case 10, or the gap between the guide 60 and the liquid 90. However, when the size of each of the gaps is equal to or smaller than the average value of the pore diameters of the filter 50, most of the air taken in from the air inlet 11 into the case 10 passes through the filter 50. Accordingly, contact between the air that has passed through the filter 50 and the liquid 90 can be promoted.

The guide 60 is formed of for example, a resin material, such as acryl, metal, or a metallic alloy, such as stainless steel. The guide 60 may be arranged so as to be in intimate contact with the filter 50 using for example, a member with urging force, such as a spring.

In the example illustrated in FIG. 2A, the shape of the filter 50 is approximately rectangular, and the guide 60 is a frame with an inverted U-shape, which is provided along three sides of the edge of the filter 50. In the example, the edge of the filter 50 on the lower side is arranged so as to be in contact with the liquid 90 in the tank 30. Accordingly, a gap that allows gas to move from the first space 13 to the second space 14 is not present under the filter 50. Thus, it may be sufficient for the guide 60 to be in contact with the inside face of the case 10 along the portions other than the edge of the filter 50 on the lower side.

The shape of the guide 60 is not particularly limited only when the guide 60 is arranged so as to block the gap or gaps. For example, the air purifier 1 may include a guide 60a illustrated in FIG. 2B.

FIG. 2B illustrates another example of the partition of the air purifier 1. For example, the left-hand illustration of FIG. 2B is a front view when the partition is viewed in a direction in which gas flows. The right-hand illustration of FIG. 2B is a side view when the partition is viewed from a side. The partition in FIG. 2B includes the filter 50 and the guide 60a.

The guide 60a is a loop-like frame provided for the entire perimeter of the edges of the filter 50 and the outside edge of the frame is in contact with the inside face of the case 10 or the inside face of the tank 30 for the entire perimeter.

Although in each of the examples illustrated in FIGS. 2A and 2B, the tank 30 is arranged so that there is no gap between the inside face of the case 10 and the outside face of the tank 30, the arrangement is not limited thereto. There may be a gap between the inside face of the case 10 and the outside face of the tank 30. In this case, the guide 60 or 60a may block the gap.

The air purifiers 1 and 1a according to the present embodiment may further include other constituents. For example, the air purifiers 1 and 1a may each include a plurality of tanks 30, a plurality of plasma generators 40, and/or a plurality of filters 50. For another example, the guide 60 or 60a may be constituted by combining a plurality of members.

### [3. Operation]

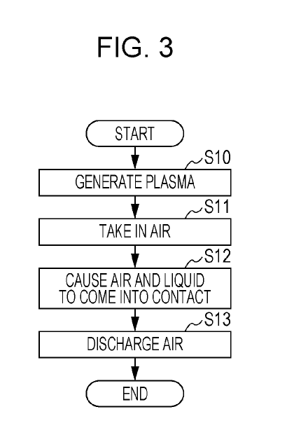

Operation of the air purifiers 1 and 1a according to the present embodiment is described with reference to FIG. 3. FIG. 3 is a flow chart that illustrates an example of the operation of the air purifiers 1 and 1a according to the present embodiment. In other words, FIG. 3 is a flow chart that illustrates an example of an air purifying method according to the present embodiment.

For example, the air purifiers 1 and 1a each start operation by the power of a main unit being turned on.

First, the plasma generator 40 generates plasma in the liquid 90 (S10). For example, the power supply 42 applies a predetermined high-voltage pulse between the pair of electrodes and thereby generates plasma in the liquid 90. A predetermined period before proceeding to a subsequent step may be set for waiting until the active species sufficiently spreads in the liquid 90 in the tank 30.

Next, the air purifiers 1 and 1a each take in air into the case 10 from the air inlet 11 (S11). For example, the intake of air is started by rotating the fan 20. Accordingly, an air flow is formed in the case 10.

After that, the air and the liquid 90 come into contact with each other (S12). For example, when the filter 50 start rotating with the pulleys 51, contact between the liquid 90 that contains the active species and the air can be promoted through the filter 50.

Lastly, the air that has been in contact with the liquid 90 is discharged from the air outlet 12 (S13).

Although FIG. 3 illustrates an example in which the steps are performed in sequence, the method is not limited thereto. For example, the steps may be performed concurrently. For example, at the time when the power of each main unit of the air purifiers 1 and 1a is turned on, all the operation of the plasma generator 40, the fan 20, and the pulleys 51 may be started. In this case, a substance in the air can be decomposed with high efficiency by causing the plasma generator 40, the fan 20, and the pulleys 51 to operate continuously.

### [4. Advantages of Decomposing Substance]

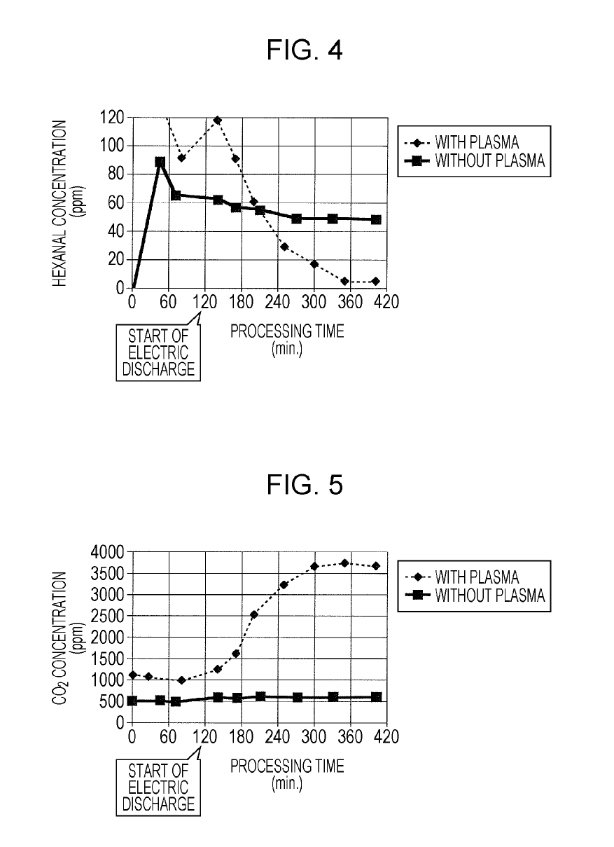

Experimental results of purifying air containing a substance to be decomposed using the air purifier 1 are described with reference to FIGS. 4 and 5.

In this experiment, hexanal (C₆H₁₂O), a kind of a chain aliphatic aldehyde, was contained in the air as the substance to be decomposed. The hexanal is an example of an odorous substance that causes an offensive odor. For example, the hexanal is produced when a fatty acid contained in oil and fat undergoes oxidation. When the hexanal is decomposed, carbon dioxide (CO₂) is produced ultimately.

The dashed lines in FIG. 4 indicate temporal change in hexanal concentration when the air purifier 1 is operated. The dashed lines in FIG. 5 indicate temporal change in carbon dioxide concentration when the air purifier 1 is operated. In each of FIGS. 4 and 5, the electric discharge starts after a lapse of 120 minutes to generate plasma.

The solid line in FIG. 4 indicates temporal change in hexanal concentration when no electric discharge is performed. The solid line in FIG. 5 indicates temporal change in carbon dioxide concentration when no electric discharge is performed.

As indicated by the dashed lines in FIG. 4, as time elapses after the generation of plasma, the hexanal concentration largely decreases. As indicated by the dashed lines in FIG. 5, as time elapses after the generation of plasma, the carbon dioxide concentration largely increases.

It is found from this that, because of the generation of plasma, the hexanal is decomposed and carbon dioxide is produced.

As indicated by the solid line in FIG. 4, without generation of plasma, the hexanal concentration hardly changes even when time elapses. As indicated by the solid line in FIG. 5, without generation of plasma, the carbon dioxide concentration hardly changes even when time elapses. Although the hexanal concentration slightly decreases, it is conceivable that this happens because part of the hexanal is decomposed into carbon dioxide by natural oxidation or taken into the liquid.

From the above-described results, it is found that the air purifier 1 according to the present embodiment can decompose hexanal with high efficiency.

### [5. Advantages of Inhibiting Discharge of Reactive Gas]

Advantages of the air purifier 1 according to the present embodiment inhibiting discharge of reactive gas are described in comparison with a reference example.

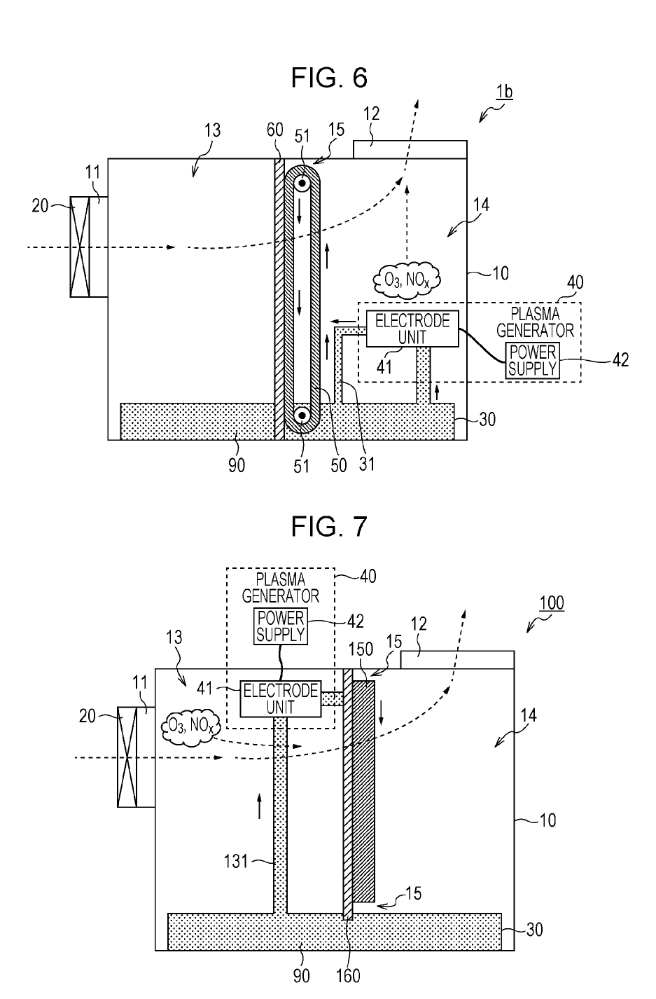

FIG. 6 illustrates a structure of an air purifier 1b according to the reference example. As illustrated in FIG. 6, compared to the air purifier 1 illustrated in FIG. 1A, the air purifier 1b according to the reference example is different in that the plasma generator 40 is arranged in the second space 14.

As described above, when the plasma generator 40 generates plasma, reactive gas, such as ozone gas or NOₓ, is produced. The reactive gas is conveyed by an air flow from the air inlet 11 to the air outlet 12.

Since the plasma generator 40 according to the reference example is arranged in the second space 14, the reactive gas is produced in the second space 14 and flows outside the case 10 from the air outlet 12 without passing through the filter 50.

In contrast, since the plasma generator 40 according to the present embodiment is arranged in the first space 13, the reactive gas is generated in the first space 13 and after passing through the filter 50, flows outside the case 10 from the air outlet 12. At the time, the filter 50 not only causes the air taken in from the air inlet 11 and the liquid 90 to come into contact with each other but also causes the reactive gas produced in the first space 13 and the liquid 90 to come into contact with each other. Thus, the reactive gas produced by the plasma generator 40 can dissolve in the liquid 90 through the filter 50. As a result, the reactive gas can be inhibited from flowing outside the case 10.

For example, ozone gas is caught into the filter 50 to dissolve in the liquid 90. The dissolved ozone can contribute to the decomposition of the substance. For example, NOₓ is caught into the filter 50 to dissolve in the liquid 90. The dissolved NOₓ becomes a nitrous acid or a nitric acid, which contributes to the decomposition of the substance. Thus, the reactive gas can promote the decomposition of the substance by dissolving in the liquid 90.

### Second Embodiment

An air purifier 100 according to a second embodiment is described with reference to FIG. 7. FIG. 7 illustrates an example of a structure of the air purifier 100 according to the present embodiment.

Compared to the air purifier 1 illustrated in FIG. 1A, the air purifier 100 illustrated in FIG. 7 is different in that piping 131, a filter 150, and a guide 160 are included instead of the piping 31, the filter 50, and the guide 60. The differences are mainly described below.

The piping 131 supplies liquid 90 stored in a tank 30 to the filter 150. For example, the piping 131 is constituted of a tubular member, such as a pipe, a tube, or a hose. For example, a pump (not illustrated) may be provided on a path of the piping 131. For example, the pump may suck up the liquid 90 from the tank 30 to supply the liquid 90 to an upper portion of the filter 150.

The piping 131 is provided with a plasma generator 40. The plasma generator 40 generates plasma to produce an active species in the liquid 90 that flows in the piping 131. Thus, the liquid 90 that contains the active species is supplied to the filter 150. Accordingly, the decomposition efficiency of the substance in the filter 150 can be enhanced.

The filter 150 is provided between an air inlet 11 and an air outlet 12. The liquid 90 and the air come into contact with each other through the filter 150. In the example illustrated in FIG. 7, the liquid 90 is supplied to the upper portion of the filter 150 through the piping 131. In FIG. 7, the air purifier 100 does not include the pulleys 51 and the filter 150 does not rotate. Thus, more various materials can be utilized as the filter 150. For example, the filter 150 may be a collection of a plurality of beads or granular substances.

Since in the example illustrated in FIG. 7, the filter 150 is stationary, the filter 150 and another part, which is the inside face of the case 10 or the guide 160 for example, can easily come into intimate contact with each other so that a gap therebetween can be made small.

The liquid 90 supplied to the upper portion of the filter 150 flows to the tank 30 along the filter 150. After that, the liquid 90 collected in the tank 30 is supplied again to the upper portion of the filter 150 through the piping 131. That is, the air purifier 100 has a circulation path, including the tank 30, the piping 131, and the filter 150, through which the liquid 90 containing the active species circulates. Thus, a substance in the air can be taken into the liquid efficiently and then be decomposed with high efficiency.

Since in the present embodiment, the liquid 90 is supplied from the upper portion of the filter 150, direct contact of the filter 150 with the liquid 90 in the tank 30 may not be required. In the example illustrated in FIG. 7, the filter 150 is arranged so as to be spaced from the liquid 90 in the tank 30. For example, the lower end of the filter 150 and the surface of the liquid 90 in the tank 30 are spaced from each other across a gap 15.

The guide 160 is arranged so as to block the gap 15 along an edge of the filter 150. The functions of the guide 160 are the same as those of the guide 60 according to the first embodiment, for example.

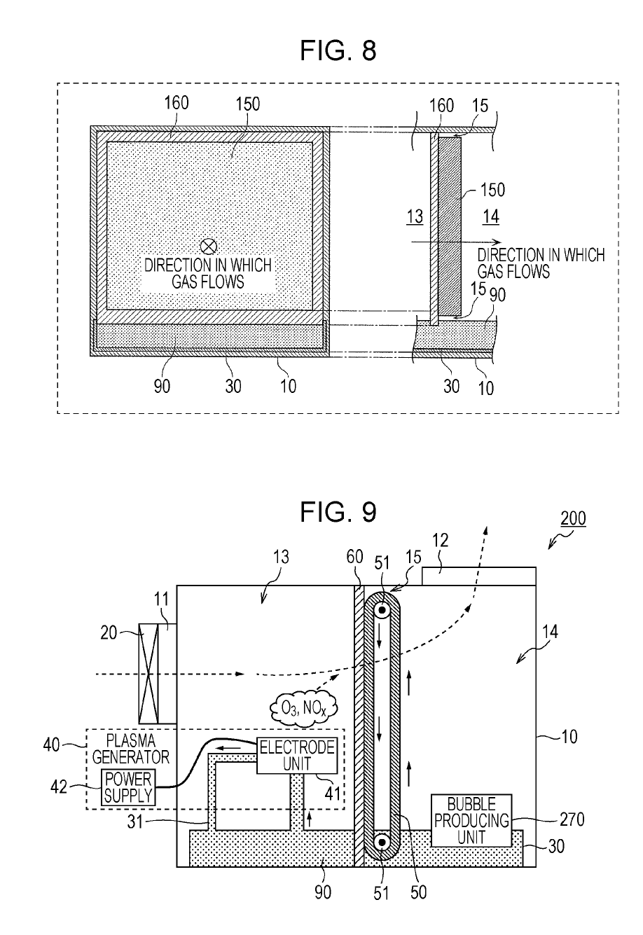

FIG. 8 illustrates an example of the partition of the air purifier 100. The left-hand illustration of FIG. 8 is a front view when the partition is viewed in a direction in which gas flows. The right-hand illustration of FIG. 8 is a side view when the partition is viewed from a side. The partition in FIG. 8 includes the filter 150 and the guide 160.

The guide 160 is a loop-like frame provided for the entire perimeter of the edges of the filter 50. In the example illustrated in FIG. 8, the gap 15 is present between the lower end of the filter 150 and the surface of the liquid 90 in the tank 30, and the guide 160 is provided so as to block the gap 15. For example, the guide 160 is arranged so as to be in contact with the lower end of the filter 150 and the liquid 90 in the tank 30.

### Third Embodiment

An air purifier 200 according to a third embodiment is described with reference to FIG. 9. FIG. 9 illustrates an example of a structure of the air purifier 200 according to the present embodiment.

Compared to the air purifier 1 illustrated in FIG. 1A, the air purifier 200 illustrated in FIG. 9 is different in that a bubble generator 270 is further included. The differences are mainly described below.

The bubble generator 270 produces nanobubbles and/or microbubbles in liquid 90.

The nanobubbles and the microbubbles are fine bubbles. For example, a bubble with a diameter of 1 μm or smaller is referred to as the nanobubble and a bubble with a diameter of 50 μm or smaller is referred to as the microbubble.

Although in the example illustrated in FIG. 9, according to the present embodiment, the bubble generator 270 is arranged in a second space 14, the arrangement is not limited thereto. The bubble generator 270 may be arranged in a first space 13 and for example, may be arranged so as to produce nanobubbles and/or microbubbles in piping 31.

At the time, the bubble generator 270 may produce the nanobubbles and/or the microbubbles from the bubble which has been produced through electric discharge. For example, the bubble generator 270 causes the produced bubble and the liquid 90 to turn in the piping 31 at a high speed, such as 400 to 600 revolutions per second. Thus, the bubble is minutely pulverized, and therefore the nanobubbles and/or the microbubbles are produced. For example, the production of the nanobubbles and/or the microbubbles may be started before the plasma generator 40 starts the electric discharge, or may be started concurrently with the start of the electric discharge.

The produced nanobubbles and/or microbubbles can adsorb the substance to be decomposed, thereby enabling the intake of the substance into the liquid 90 to be promoted. Thus, reaction between the substance and an active species in the liquid 90 can easily occur, thereby enhancing the decomposition efficiency. In addition, since the reactive gas produced by the plasma generator 40, such as ozone gas or NOₓ, can easily dissolve in the liquid 90, the reactive gas can be inhibited from flowing outside a case 10.

### Fourth Embodiment

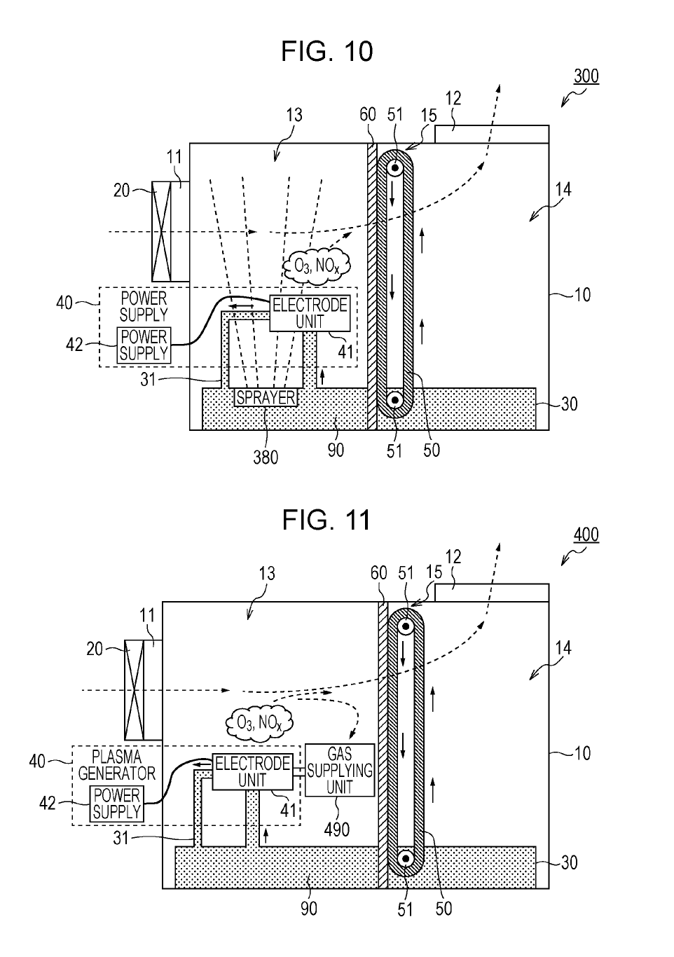

An air purifier 300 according to the fourth embodiment is described with reference to FIG. 10. FIG. 10 illustrates an example of a structure of the air purifier 300 according to the present embodiment.

Compared to the air purifier 1 illustrated in FIG. 1A, the air purifier 300 illustrated in FIG. 10 is different in that a sprayer 380 is further included. The differences are mainly described below.

That is, the sprayer 380 sprays liquid 90 in a tank 30 into a first space 13 in a mist state. The first space 13 is a space where the air taken in from an air inlet 11 builds up or moves before coming into contact with a filter 50.

For example, the sprayer 380 includes a spray nozzle. In this case, the sprayer 380 sprays the liquid 90 in the first space 13 while the liquid 90 is changed into mist of approximately several microns to several tens of microns.

For example, the spray of the liquid 90 may be started before the air inlet 11 starts taking in air (S11 in FIG. 3), or may be started concurrently with the start of the intake of the air. For example, the spray of the liquid 90 may be started after the plasma generator 40 starts the electric discharge (S10 in FIG. 3), or may be started concurrently with the start of the electric discharge.

When the liquid 90 is sprayed in the air in the first space 13, the liquid 90 in the mist state can easily come into contact with the substance in the air, so that the substance can be easily taken into the liquid 90. After the liquid 90 containing the substance is collected by the filter 50, the substance can be effectively decomposed.

If the liquid 90 in the mist state contains an active species sufficiently, the substance can be decomposed when taken into the liquid 90 in the mist state. Accordingly, the decomposition efficiency of the substance can be further enhanced. In addition, the liquid 90 in the mist state can promote dissolution of the reactive gas which has been produced by the plasma generator 40, such as ozone gas or NOₓ, into the liquid 90. Thus, the reactive gas can be inhibited from flowing outside a case 10.

Although in the example illustrated in FIG. 10, the sprayer 380 sprays the liquid 90 upward from a lower portion of the case 10, the sprayer 380 may spray the liquid 90 downward from an upper portion of the case 10. The spraying method may be any method.

### Fifth Embodiment

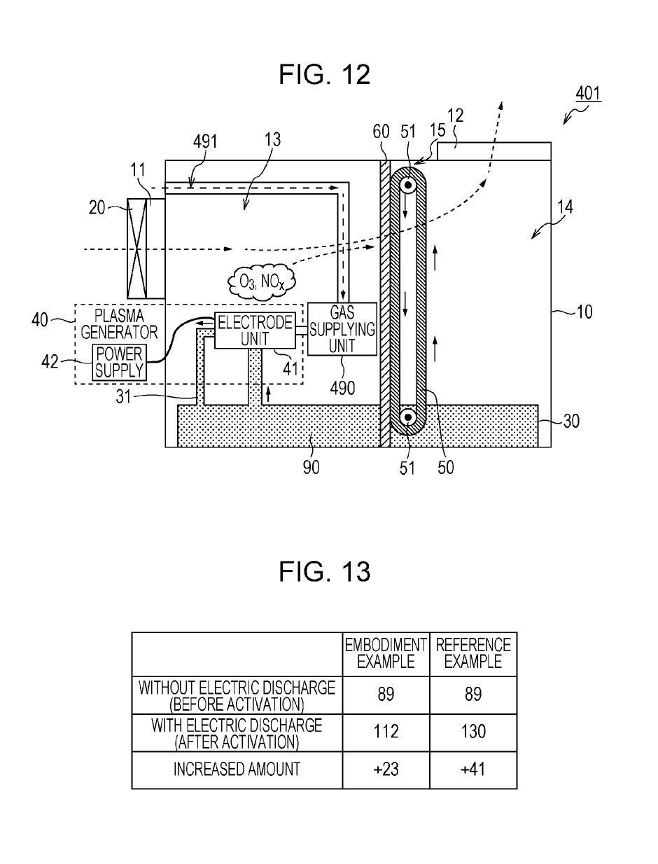

Air purifiers 400 and 401 according to the fifth embodiment is described with reference to FIGS. 11 and 12. FIGS. 11 and 12 illustrate examples of structures of the air purifiers 400 and 401 according to the present embodiment, respectively.

Compared to the air purifier 1 illustrated in FIG. 1A, the air purifier 400 illustrated in FIG. 11 is different in that a gas feeder 490 is further included. The differences are mainly described below.

The gas feeder 490 is, for example, a pump. In the example illustrated in FIG. 11, the gas feeder 490 supplies part of the air taken in from an air inlet 11 into liquid 90 stored in a tank 30. Thus, for example, a bubble is produced in the proximity of an electrode unit 41 of a plasma generator 40, such as a pair of electrodes. The bubble may be larger than nanobubbles and/or microbubbles, such as one large bubble that can be sufficiently identified with a naked eye.

For example, the plasma generator 40 causes electric discharge in the bubble so as to generate plasma therein . For example, the gas feeder 490 may start supplying air before the start of the electric discharge of the plasma generator 40 (S10 in FIG. 3), or may start supplying air concurrently with the start of the electric discharge.

When at the time, the gas feeder 490 supplies a minute bubble in addition to the large bubble, the plasma can be included in the minute bubble. Thus, an active species, such as a hydroxyl radical, can be produced in the proximity of the minute bubble.

In the example illustrated in FIG. 11, the gas feeder 490 is arranged in a first space 13. When the gas feeder 490 takes in the air containing the substance to be decomposed, plasma is generated in a bubble containing the substance. As a result, the substance can come into direct contact with the reactive gas produced using plasma and thus can be decomposed high efficiently.

Compared to the air purifier 400 illustrated in FIG. 11, the air purifier 401 illustrated in FIG. 12 is different in that a gas leading-in pipe 491 is included. For example, the gas feeder 490 illustrated in FIG. 12 takes in air from the outside of a case 10 through the gas leading-in pipe 491 and then supplies the air to the electrode unit 41 of the plasma generator 40, which is a pair of electrodes for example.

### Supplement

Experimental results of measuring the concentration of an odorous substance in the proximity of the air outlet 12 using a volatile organic compound (VOC) sensor in an example of each of the air purifiers according to the second embodiment and the fifth embodiment are described below with reference to FIG. 13. FIG. 13 illustrates the concentrations of the odorous substance in the proximity of each air outlet of the air purifiers according to the example and the reference example, which are sensed using the VOC sensor. In the air purifier according to the example, on the basis of the air purifier 100 illustrated in FIG. 7, the gas feeder 490 and the gas leading-in pipe 491 illustrated in FIG. 12 are further added. The air purifier according to the reference example is the air purifier 1b illustrated in FIG. 6.

As illustrated in FIG. 13, when there is no electric discharge (i.e., before the activation of the air purifier), the values of the VOC sensors according to the example and the reference example are the same.

In contrast, when plasm is generated (i.e., after the plasma generator 40 is activated), the value of the VOC sensor in the proximity of each air outlet 12 increases. This is caused by the gas produced by the plasma generator 40, such as ozone gas or NOₓ. However, the increased amount of the value of the VOC sensor according to the example is approximately half of that according to the reference example. That is, the air purifier according to the example can suppress the increased amount of the odorous substance produced by the plasma generator 40.

The results indicate that when the plasma generator 40 is arranged in the first space 13, the gas produced by the plasma generator 40, such as ozone gas or NOₓ, is effectively removed by the filter 50.

### Other Embodiments

The air purifiers and the air purifying methods according to various embodiments are described above as examples. However, the present disclosure is not limited to these embodiments. The present disclosure includes what is obtained by adding a change with which a person skilled in the art can come up and what is obtained by combining constituents of different embodiments as long as the gist of the present disclosure is not departed.

For example, although the above-described embodiment presents an example in which the liquid 90 is supplied to the filter 50 by rotating the filter 50 and an example in which the liquid 90 is supplied to the filter 150 through the piping 131, the method of supplying the liquid to the filter is not limited thereto. For example, the air purifier may include a capillary as an example of the gas-liquid contact member. The capillary may suck up the water in the tank by utilizing a capillary phenomenon.

For example, although in the above-described embodiment, the guide is arranged on the side of the first space with respect to the filter, the arrangement is not limited thereto. The guide may be arranged on the side of the second space with respect to the filter. For another example, the guide may be arranged in the same plane as the filter. For example, the guide may be arranged so as to fill a gap between an edge of the filter and the inside face of the case.

For example, although in the above-described embodiment, the guide is arranged so that 90% or more of the gas that moves from the first space to the second space passes through the filter, the arrangement is not limited thereto. It is sufficient that the guide blocks at least part of various gaps and the gas that passes through the filter may be less than 90%.

For example, although in the above-described embodiment, the inner space of the case 10 is partitioned into the first space and the second space by the filter and the guide, the arrangement is not limited thereto. For example, when the proportion of the gas that passes through the filter in the gas that moves from the first space to the second space satisfies a desired proportion, such as 90% or more, the inner space of the case 10 may be partitioned into the first space and the second space by the filter only. In other words, the partition that partitions the inner space of the case 10 into the first space and the second space may be the filter only. That is, the guide has a given structure.

For example, when the proportion of the gas that passes through the filter in the gas that moves from the first space to the second space satisfies a desired proportion, such as 90% or more, the air purifier may include another channel through which the gas returns from the second space to the first space without passing through the filter.

For example, the arrangements of the air inlet, the air outlet, the tank, and the filter are not particularly limited. For example, the air inlet may be positioned on the lower face of the case and the air outlet may be positioned on the upper face of the case. In this case, the filter may be arranged laterally so as to face the upper face of the case.

For example, the filter 150 according to the second embodiment described above may be in direct contact with the liquid 90.

In the above-described various embodiments, the air purifier may include a controller. For example, the controller may control at least one of the power supply of the plasma generator, the fan, the pulleys, the pump, the gas feeder, and the bubble generator. For example, the controller may perform the sequence indicated in FIG. 3. The controller includes, for example, non-volatile memory where a program of a predetermined sequence is stored, volatile memory, which is a transitory storage area for performing the program, an input/output port, and a processor for performing the program. The controller is for example, a microcomputer.

Each of the above-described embodiments allows various changes, replacements, additions, or omissions within the scope of aspects of the present disclosure or a scope equivalent thereto.

The air purifier of the present disclosure can be utilized for a deodorizing apparatus, a sterilizer, or an air cleaner for example.

What is claimed is:

1. An air purifier comprising:
a case having an air inlet and an air outlet;
a tank that stores liquid, the tank disposed in the case;
a fan that produces an air flow from the air inlet to the air outlet;
a partition disposed in the case, the partition including a filter through which the air flow comes into contact with the liquid; and
a plasma generator that generates plasma which is to be in contact with the liquid, the plasma generator including a pair of electrodes which is disposed in a first space between the air inlet and the partition and a power supply which applies a voltage between the pair of electrodes.

2. The air purifier according to claim 1, wherein
the partition partitions an inner space of the case into the first space and a second space between the partition and the air outlet.

3. The air purifier according to claim 2, wherein
90% or more of gas that moves from the first space to the second space passes through the filter.

4. The air purifier according to claim 2, wherein
the first space and the second space are communicated with each other through a gap between an inside face of the case and an outside edge of the partition, and
a size of the gap is equal to or smaller than an average value of diameters of pores of the filter.

5. The air purifier according to claim 1, wherein
part of an outside edge of the filter is in contact with the liquid in the tank.

6. The air purifier according to claim 1, further comprising:
piping through which the liquid is supplied from the tank to the filter.

7. The air purifier according to claim 2, wherein
the partition further includes a guide that extends along at least part of an outside edge of the filter.

8. The air purifier according to claim 7, wherein
the first space and the second space are communicated with each other through a gap between the guide and the filter, and
a size of the gap is equal to or smaller than an average value of diameters of pores of the filter.

9. The air purifier according to claim 7, wherein
the first space and the second space are communicated with each other through a gap between the guide and the liquid in the tank, and
a size of the gap is equal to or smaller than an average value of diameters of pores of the filter.

10. The air purifier according to claim 7, wherein
the guide includes a frame.

11. The air purifier according to claim 10, wherein
part of the outside edge of the frame is in contact with the liquid in the tank, and another part of the outside edge of the frame is in contact with an inside face of the case.

12. The air purifier according to claim 10, wherein
part of the outside edge of the filter is in contact with the liquid in the tank, and
the frame extends along another part of the outside edge of the filter.

13. The air purifier according to claim 10, wherein
the filter is spaced apart from the liquid in the tank,
the frame has a loop-like shape, and
part of the frame blocks a gap between the filter and the liquid in the tank.

14. The air purifier according to claim 1, further comprising:
a pump that supplies gas with which at least part of the pair of electrodes is covered.
